(19) United States
(12) Patent Application Publication
(10) Pub. No.: US 2022/0347037 A1
(43) Pub. Date: Nov. 3, 2022

(54) DEVICE FOR PERFORMING INDIVIDUAL MOVEMENT ANALYSIS AND THERAPY ON A PATIENT

(71) Applicant: ReActive Robotics GmbH, Muenchen (DE)

(72) Inventor: Alexander Koenig, Muenchen (DE)

(21) Appl. No.: 17/778,108

(22) PCT Filed: Nov. 13, 2020
(86) PCT No.: PCT/DE2020/100971

§ 371 Date: May 19, 2022

(30) Foreign Application Priority Data
Nov. 22, 2019 (DE) 10 2019 131 703.7

(51) Int. Cl. A61H 1/02; A61B 5/11; A61B 5/00

(52) U.S. Cl. CPC A61H 1/0255; A61B 5/1122; A61B 5/4836; A61H 2201/5061; A61H 2201/5069; A61H 2201/1642; A61H 2201/105; A61H 2230/605

(57) ABSTRACT

A device for performing individual movement analysis and movement therapy on a patient includes a control and analysis unit configured to control a first intervention device in such a way that a first trajectory of the movement of an extremity of the patient is disrupted by a force exerted by a movement module onto the extremity of the patient. A response to this disruption is measured by changed measured values from at least one force sensor and/or at least one angle sensor, and a new, second trajectory is calculated therefrom. New control parameters for controlling the first intervention device are calculated from a comparison of the second trajectory with the first trajectory and/or a target trajectory or the comparison of the disrupted measured values with the non-disrupted measured values.

The present invention relates to a device for performing an individual movement analysis and movement therapy on a patient, having the features of the preamble of claim 1.

Bedridden patients, especially those with neurological impairments for example following a cerebrovascular accident, following traumatic brain injuries and/or paraplegia, demonstrably profit from a timely onset of movement therapy for improving both motor and neurological abilities. In this case, the success of such movement therapy depends significantly on the individual circumstances of each individual patient, and is very difficult to estimate or evaluate, especially for patients in early stages of their healing process. For example, two patients, each with a cerebrovascular accident, who have a comparable physical impairment and comparable clinical findings, for example from MRI imaging, may recover completely differently in the case of a comparable intervention (e.g., 3 weeks' rehabilitation therapy, in each case for 1 hour a day, 5 days a week). Conventionally, patients who do not recover are referred to as “non-responders”, while patients who “come to life” in response to therapy are referred to as so-called “responders”.

Therefore, there is virtually no individualization of a therapy intervention, especially for neurological patients, since a prediction of the effectiveness/non-effectiveness is difficult at the start of the convalescence and most patients only receive the form of therapy currently possible or carried out as standard in the respective hospital. In contrast to other clinical pictures, where statistical or methodical methods that facilitate an individual healing prediction are known, such methods are virtually unknown in the case of neurological patients, which leads to therapy interventions, especially an early movement therapy, not being able to be adapted in patient-individualized fashion.

By way of example, DE 10 2017 114 290 A1 only discloses a measuring method for determining the length ratios, the position and/or the radius of movement of the lower extremities of a bedridden patient, and a device for carrying out such a measuring method. The device disclosed therein provides for, inter alia, a force sensor that monitors the force introduced into a knee orthosis by way of a cantilever and a connecting element but does not facilitate an individual healing prediction.

Taking this as a starting point, the present invention is based on the object of providing a device, improved over the prior art, for performing an individual movement analysis and movement therapy on a patient, the device in particular facilitating the testing of patients in the early stage of their healing process in respect of the chances of success of possible therapy interventions, the development of an individualized therapy strategy and the tracking of the healing process even during the movement therapy.

This object is achieved by a device having the features of independent claim 1.

In relation to devices in the prior art, the device according to the invention is distinguished in that the control and analysis unit is configured to control the first intervention means in such a way that a first trajectory of the movement of the extremity of the patient is disrupted by a force exerted by the movement module on the extremity of the patient; a response to this disruption is measured by way of altered measured values of the at least one force sensor and/or at least one angle sensor and a new, second trajectory is calculated therefrom; and new control parameters for (further) control of the first intervention means are calculated from a comparison of the second trajectory with the first trajectory and/or a target trajectory or the comparison of the disrupted and non-disrupted measured values of the at least one force sensor and/or at least one angle sensor.

Advantageously, such a device allows an individual gait analysis to be performed on a patient in the early stage of rehabilitation, in particular for a bedridden patient, and allows the results of said analysis to be considered automatically in a further (more in-depth) movement therapy performed with the aid of the first intervention means, especially with the aid of the rehabilitation mechanism. Moreover, the success of the therapy can advantageously be determined in automated fashion, with small responses by the patient to the disruptions inflicted on them by the first intervention means already being detected despite not being optically detectable by a therapist.

In this case, the disruption is introduced by way of the first intention means, especially the rehabilitation mechanism, preferably in the form of “clamp trials”, that is to say in the form of restricting the movement radius, making movement more difficult and/or preventing movement. The analysis is implemented by measuring the force exerted by the patient to counter this and/or by determining ii the deviation of various trajectories.

In this case, the measured values of each individual patient can be advantageously stored and used both for their own individual therapy or for determining their own therapy success and for the generation of a database of “typical” progresses of therapy. On account of a statistical evaluation of these data, ever more accurate predictions about different progresses of therapy can advantageously be established, even already at a time at which a therapist/physician is (still) optically unable to identify a change in the condition of the respective patient.

What has proven its worth in a preferred embodiment of the invention is that the rehabilitation mechanism has at least one knee module capable of being brought into operative connection with the knee joints of the patient as movement module; the knee module comprising at least one force sensor for measuring an absolute value of a force between the knee module and a knee joint of the patient, and/or at least one angle sensor for measuring the direction of the force between the knee module and the knee joint of the patient; and/or a foot module for accommodating the feet of the patient; the foot module comprising at least one force sensor for measuring an absolute value of a force between the foot module and each foot of the patient. A rehabilitation mechanism having at least one knee module and/or foot module as a movement module is particularly suitable for performing an individual movement analysis and movement therapy on the leg movement of a patient, with the comparatively large muscle groups of the legs advantageously being involved.

Moreover, in a preferred embodiment of the invention, the device comprises at least one second intervention means for interaction with the patient, the second intervention means being configured to interchange (control) data with the control and analysis unit. If at least one second intervention means is provided, it is possible to test and/or controllably alter the influence of various therapy components or therapy strategies and concepts—for example by the addition of one or more further stimuli—on the patient, as a result of which the individually “best”, most effective therapy performance for a patient can be determined advantageously in automated fashion.

What has proven its worth in this case is that the second intervention means is configured to interact with the body of the patient, especially with their leg, knee joint and/or foot of the patient. A second intervention means configured in this way advantageously facilitates a direct mechanical interaction with the patient or exertion of influence on the patient. To this end, the second intervention means can preferably be arranged either directly on the first intervention means, especially on the rehabilitation mechanism, or else on the device at a distance from the first intervention means.

To this end, the second intervention means can preferably be a means for generating a vibration and/or a means for performing electromyostimulation. A second intervention means thus embodied advantageously facilitates a mechanical or electrical or other type of external muscle stimulation.

In a further preferred embodiment, the second intervention means may also be a means for generating visual stimuli and/or a means for generating acoustic stimuli. Firstly, this advantageously facilitates a visual and/or acoustic stimulation of the patient but secondly also offers the option of providing the patient with a type of visual/acoustic feedback in relation to their performance.

Moreover, an embodiment of the invention in which the second intervention means is a means for scheduled administration of a medicament has proven its worth. A means for scheduled administration of a medicament advantageously facilitates the controlled change of a pharmacological intervention on the patient. The provision of a second intervention means thus embodied makes it possible to test which medicaments (or the omission of which medicaments) can advantageously influence the progress of the rehabilitation, that is to say whether for example a different sedation medicament or a certain neurotransmitter such as serotonin, noradrenaline or dopamine should be chosen on an individual basis for the purposes of increasing the effectivity of the movement therapy.

ii What has proven its worth in a further preferred embodiment is that the control and analysis unit comprises a means for performing electromyography. If the control and analysis unit comprises a means for performing electromyography, it is advantageously already possible to measure small changes in the electrical muscular activity of a patient, allowing a test on the state of health, especially in the case of unconscious patients shortly after a cerebrovascular accident.

Finally, an embodiment of the device according to the invention in which the device comprises a device for reversibly connecting the device, especially the first intervention means, to a bed, in particular to a hospital bed that can be driven into the vertical, has proven its worth. A device for reversibly connecting the device, especially the first intervention means, to a bed, in particular to a hospital bed that can be driven into the vertical, advantageously facilitates the mobile embodiment of the device according to the invention or the retrofitting of conventional beds, especially hospital beds, with a device according to the invention, and in this way advantageously reduces the procurement costs for hospitals and/or care homes. A possible device for reversible connection is disclosed in DE 10 2018 129 370.4 from the applicant, for example.

These and additional details and further advantages of the invention are described below on the basis of preferred exemplary embodiments, which, however, do not restrict the present invention, and in conjunction with the attached drawing, in which schematically:

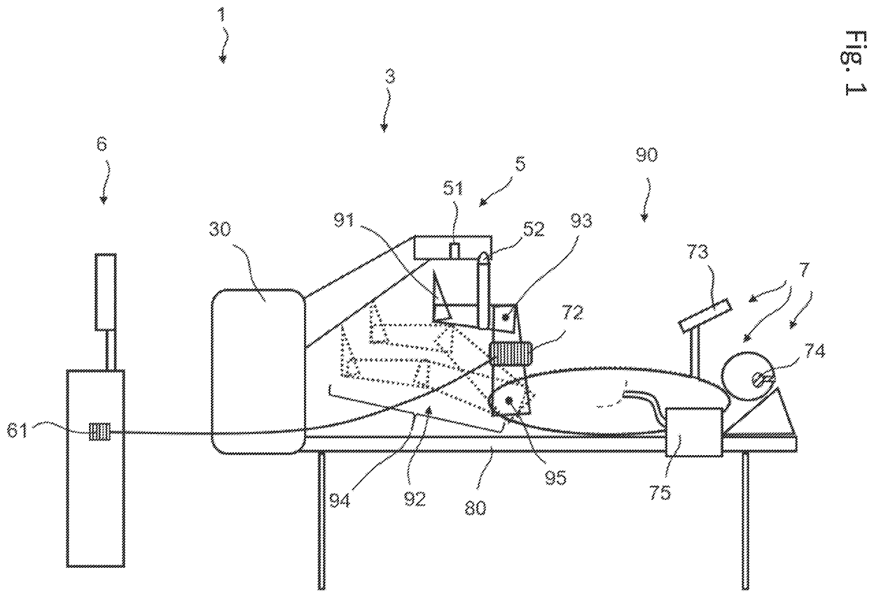

FIG. 1 shows a side view of an embodiment of a device according to the invention;

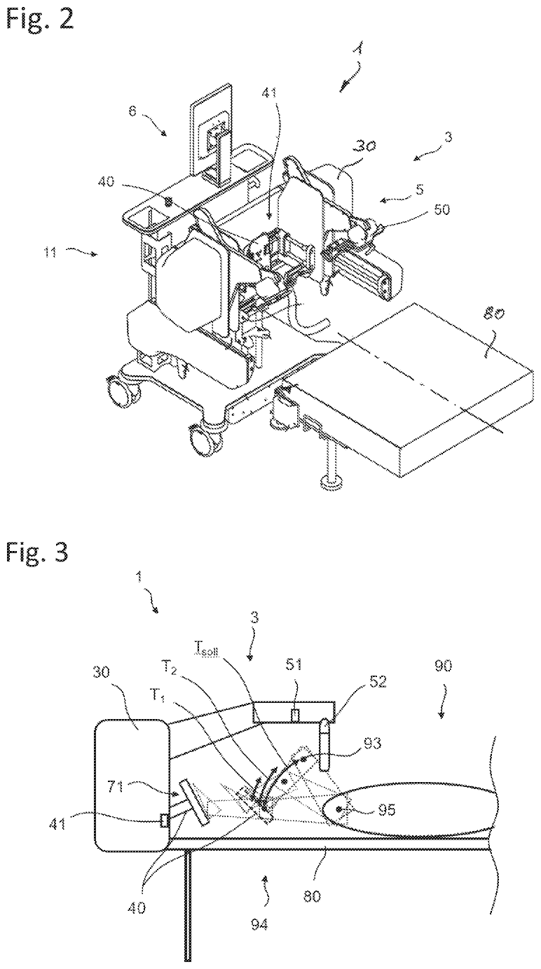

FIG. 2 shows a further embodiment of a device according to the invention, having a device for reversibly connecting the device to a bed;

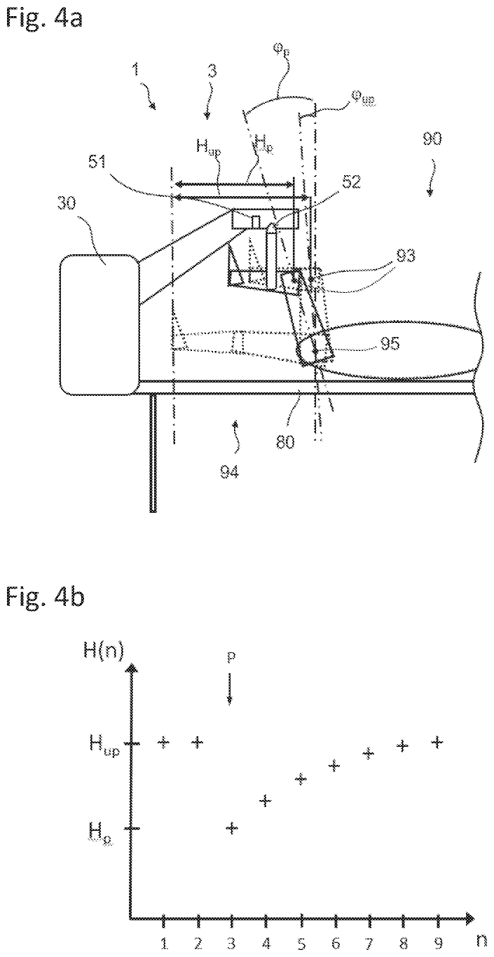

FIG. 3 shows a further embodiment of the device according to the invention while carrying out an individual movement analysis and movement therapy, with trajectories plotted in exemplary fashion;

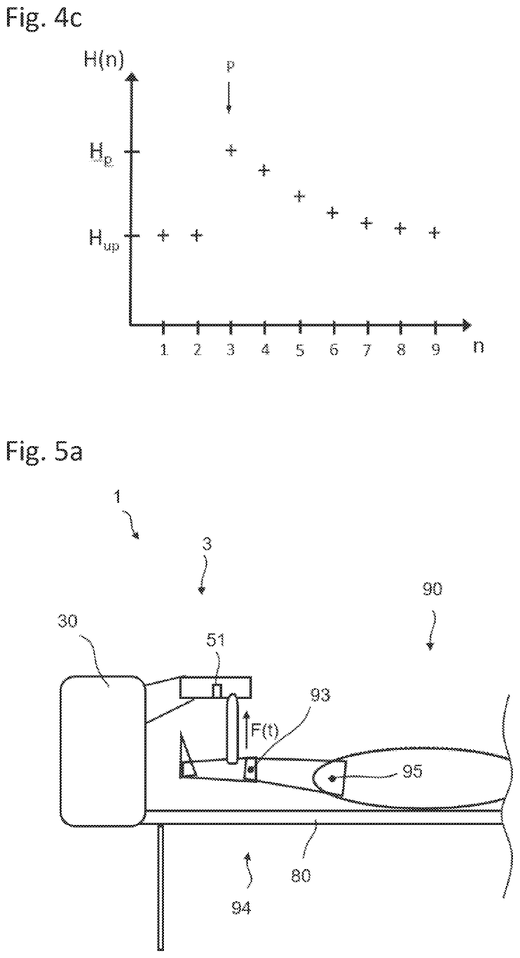

FIG. 4a shows a further embodiment of the device according to the invention while carrying out an individual movement analysis and movement therapy, with step heights and flexion angles of a hip joint plotted in exemplary fashion;

FIG. 4b shows an exemplary plot of the step heights measured by the device from FIG. 4a before and after a disruption, for a first case where the step height is greater before the disruption than after the disruption;

FIG. 4c shows an exemplary plot of the step heights measured by the device from FIG. 4a before and after a disruption, for a second case where the step height is smaller before the disruption than after the disruption;

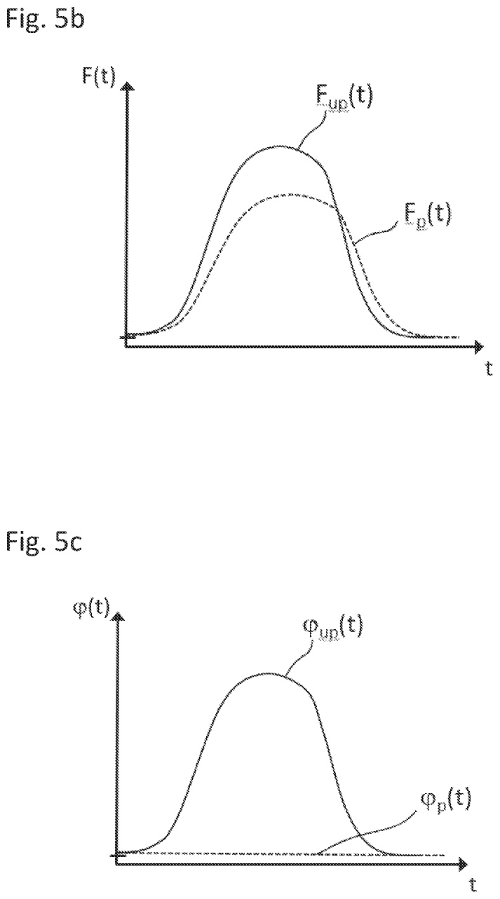

FIG. 5a shows a further embodiment of the device according to the invention while carrying out an individual movement analysis and movement therapy, with the disruption consisting of the device preventing a movement of the extremity;

FIG. 5b shows an exemplary plot of the force exerted by the patient on the device from FIG. 5a, as measured by the latter, in the case of a non-disrupted movement of the extremity and in the case of a disrupted, especially fully prevented, movement of the extremity; and

FIG. 5c shows an exemplary plot of a flexion angle, corresponding to the performance of an individual movement analysis and movement therapy as shown in FIGS. 5a and 5b in exemplary fashion, as a function of time.

In the following description of preferred embodiments of the present invention, identical reference signs denote identical or comparable components.

FIG. 1 shows a side view of an embodiment of a device 1 according to the invention. The device 1 according to the invention comprises at least one first intervention means 3 in the form of a rehabilitation mechanism 30, suitably designed for an automated rehabilitation movement of the extremities 94, at least ii of the joints, muscles and tendons of the legs 92, of a patient 90 according to plan, having at least one movement module 5 capable of being brought into operative connection with the extremities 94 of the patient 90. In this case, the movement module 5 comprises at least one force sensor 41; 51 for measuring an absolute value of a force F(t) between the movement module 5 and an extremity 94 of the patient 90 (cf. also FIGS. 3 and 5A) and/or at least one angle sensor 52 for measuring the direction of the force F(t) between the movement module 5 and the extremity 94 of the patient 90. The device 1 according to the invention moreover comprises at least one control and analysis unit 6 for controlling the first intervention means 3 and analysis of a trajectory T₁; T₂; . . . of the movement of the extremity 94 of the patient 90 calculated from the actual values of the at least one force sensor 41; 51 and/or at least one angle sensor 52,

The control and analysis unit 6 of the device 1 according to the invention is configured to control the first intervention means 3 in such a way that a first trajectory T₁ of the movement of the extremity 94 of the patient 90 is disrupted by a force exerted by the movement module 5 on the extremity 94 of the patient 90; a response to this disruption P is measured by way of altered measured values of the at least one force sensor 41; 51 and/or at least one angle sensor 52 and a new, second trajectory T₂ is calculated therefrom; and new control parameters for (further) control of the first intervention means 3 are calculated from a comparison of the second trajectory T₂ with the first trajectory T₁ and/or a target trajectory Tₛₒₗₗ or the comparison of the disrupted and non-disrupted measured values of the at least one force sensor 41; 51 and/or at least one angle sensor 52. The force sensor or force sensors 41; 51 can be arranged, in particular, on a cantilever of the movement module 5, as shown in FIG. 1, and/or in a region near the feet 91 of the patient 90, especially on a foot module 40 of the rehabilitation mechanism 30 (cf. FIG. 2 and FIG. 3 as well).

FIG. 1 moreover shows that the device 1 preferably also may comprise at least one second intervention means 7 for interaction with the patient 90, the second intervention means 7 preferably being configured to interchange (control) data with the control and analysis unit 6. In particular, the second intervention means 7 can be configured to interact with the body of the patient 90, especially with their leg 92, knee joint 93 and/or foot 91, and thereby for example to generate a further mechanical disruption P to the movement of the extremity 94 by exerting a force on the extremity 94 of the patient 90. To this end, the second intervention means 7 can be, in particular, a means 71 for generating a vibration (cf. FIG. 3) and/or a means 72 for performing electromyostimulation. Preferably, the control and analysis unit 6 may in the latter case also comprise a means 61 for performing electromyography, which advantageously serves to control and evaluate electrical or other types of external muscle stimulations. The second intervention means 7 may also be, alternatively or cumulatively, a means 73 for generating visual stimuli and/or a means 74 for generating acoustic stimuli, as shown. In such an embodiment of the invention, the second intervention means 7 can advantageously act on other senses—sense of vision and/or sense of hearing—of the patient 90 as a disruption P. Particularly in the case of patients 90 with neurological damage, the action via different sensory organs may lead to better (brain) stimulation, which in turn may advantageously assist the progress of the rehabilitation. The device 1 can then advantageously measure and evaluate the influence of such disruptions P, especially acoustic and/or visual disruptions, on the patient 90, as described above. Finally, the second intervention means 7 may, alternatively or cumulatively, also be a means 75 for scheduled administration of a medicament. In this case, the disruption P consists in a targeted administration and/or the targeted omission of a medicament, that is to say a pharmacological intervention. By way of example, such a second intervention means 7 advantageously facilitates testing of which medicaments are advantageous or disadvantageous for the healing process, what active ingredient doses are effective and whether, for example, a change of sedative or the administration of a certain neurotransmitter such as serotonin, noradrenaline or dopamine can influence the healing process (preferably in a positive manner). The device 1 according to the invention can advantageously measure deviations in the behavior of the patient, in particular deviations in their sequence of movement, which are so small that they would not (yet) even be noticed by a therapist during a conventional movement therapy, as a result of which the accuracy of the results obtained advantageously increases.

FIG. 2 shows a further embodiment of a device 1 according to the invention, which comprises a device 11 for reversibly connecting the device 1, especially the first intervention means 3, to a bed 80. In this case, the bed 80 can preferably be a hospital bed that can be driven into the vertical. As is shown in FIG. 2, the device 1 can have a mobile design with the aid of such a device 11 for a reversible connection, and can be connected to a bed 80 for carrying out an individual movement analysis and movement therapy and can be removed again after the completion of the analysis or therapy. A possible device 11 is disclosed for example in DE 10 2018 129 370.4 from the applicant, comprehensive reference being made thereto at this juncture in respect of the functionality of the device 11.

FIG. 2 moreover shows that the device 1 may also have a rehabilitation mechanism 30 as a movement module 5, said rehabilitation mechanism comprising at least one knee module 50 capable of being brought into operative connection with the knee joints 93 of the patient 90; the knee module 50 preferably comprising at least one force sensor 51 for measuring an absolute value of a force F(t) between the knee module 50 and a knee joint 93 of the patient 90 and/or at least one angle sensor 52 for measuring the direction of the force F(t) between the knee module 50 and the knee joint 93 of the patient 90. Alternatively or cumulatively, the rehabilitation mechanism 30 may also comprise a foot module 40 for accommodating the feet 91 of the patient 90; the foot module 40 preferably comprising at least one force sensor 41 for measuring an absolute value of a force between the foot module 40 and each foot 91 of the patient 90.

FIG. 3 now shows a further embodiment of the device 1 according to the invention while carrying out an individual movement analysis and movement therapy, with trajectories Tₛₒₗₗ, T₁, T₂ plotted in exemplary fashion. To carry out a movement analysis, extremities 94 of the patient 90, shown here using the example of a leg 92, may be brought into operative connection with the movement module 5. To this end, FIG. 3 shows an operative connection on the thigh in the region of the knee joint 93 in exemplary fashion; however, the leg 92 may also be brought into operative connection with the first intervention means 3 on the lower leg, as shown in FIGS. 1, 4a and 5a, and/or at another location. With the aid of the at least one force sensor 41; 51—FIG. 3 shows a force sensor 51 on a cantilever of the movement module 5 and a force sensor 41 in the region of a foot module 40—and/or the at least one angle sensor 52, it is then possible to determine a trajectory T₁; T₂; . . . by means of the control and analysis unit 6 by way of the measurement of an absolute value of a force F(t) between the movement module 5 and the extremity 94 or by measuring the direction of the force F(t) during a movement of the extremity 94, and to store said trajectory for further use. In this way, the device 1 for example advantageously facilitates the creation of a database of non-disrupted, “normal” trajectories T₁; T₂; . . . of healthy humans, with the first intervention means 3, in particular the rehabilitation mechanism 30, not exerting any sort of disruption P on the movement of the subject during such a recording, but instead only accompanying the movement.

In the case of a convalescing patient 90, an actual trajectory T₁; T₂; . . . of the movement of the extremities 94 firstly can be measured in a similar manner with the aid of the device 1 according to the invention and can be compared with the above-described database entries, that is to say with target trajectories Tₛₒₗₗ of healthy humans, for the purposes of estimating the current healing state. Secondly, a disruption P that acts on the patient 90 can also be generated by means of the first intervention means 3, especially by means of the movement module 5, and/or by means of one or more second intervention means 7, before and/or during a movement of the extremities 94, and a possible deviation from a comparison trajectory induced thereby can be measured. In this case, in particular, the comparison trajectory can be either a target trajectory Tₛₒₗₗ of a healthy human stored in the database or else a trajectory T₁; T₂; . . . of the patient 90 themselves already measured earlier, hence advantageously logging the progress of the healing of the respective patient 90 on an individual basis.

The device 1 according to the invention moreover allows statements to be made about the ability of the patient 90 to adapt to a disturbance P by way of a quantification of non-disrupted and disrupted measured values of the at least one force sensor 41; 51 and/or at least one angle sensor 52, and thereby allows an assessment of said patient's state of health and/or the rehabilitation success. Two of these quantification methods are presented below in exemplary fashion:

FIG. 4a shows an embodiment of the device 1 according to the invention while performing an individual movement analysis and movement therapy, with exemplary plotted step heights Hᵤₚ; Hₚ and flexion angles φᵤₚ; φₚ of a hip joint 95. In this quantification method, it is possible to generate a disruption P acting on the patient 90 during a plurality of successive movement cycles (“steps”) controlled and/or accompanied by the device 1, be it by exerting a force by way of the first intervention means 3 or by another disruption P by way of a second intervention means 7. In this case, a step height Hᵤₚ; Hₚ and/or a flexion angle φᵤₚ; φₚ of a hip joint 95 can preferably be chosen as measurement parameter, with the disruption P, especially an exertion of force on the extremities 94 of the patient 90, being able to have effects on the measured step height Hᵤₚ; Hₚ or the flexion angles φᵤₚ; φₚ of a hip joint 95. Depending on the state of health of the patient 90, there may be a motor adaptation of the patient 90 after a certain period during which the disruption P acts on the patient 90 and/or after the disruption P was removed again; the patient 90 recovers from the respective disruption P, which in turn may be reflected in a change of the aforementioned measured values.

FIG. 4b shows an exemplary plot of the step heights Hᵤₚ; Hₚ measured by the device 1 from FIG. 4a before and after a disruption P for a first case, where the step height Hᵤₚ before the disruption P is greater than the step height Hₚ after the disruption P.

Accordingly, FIG. 4c shows an exemplary plot of the step heights Hᵤₚ; Hₚ measured by the device 1 from FIG. 4a before and after a disruption P for a second case, where the step height Hᵤₚ before the disruption P is smaller than the step height Hₚ after the disruption. In both figures, H(n) denotes the step height of the respective step numbered n.

It is evident from both FIG. 4b and FIG. 4c that the step height Hₚ after the onset of the disruption P differs significantly from the non-disrupted step height Hᵤₚ. As the number n of steps increases following the onset of the disruption P, the measured value approaches the non-disrupted step height Hᵤₚ again, however, the speed with which and/or the extent to which this adaptation occurs (=the “ability to recover”) depending on the current, individual state of health of the patient 90 and therefore being able to provide indications in regard to the effectiveness of an applied form of therapy (movement therapy, medicinal therapy, etc.).

A second possible quantification process facilitated by the device 1 according to the invention is outlined in FIGS. 5a to c.

FIG. 5a shows one embodiment of the device 1 according to the invention while carrying out an individual movement analysis and movement therapy, with the disruption P consisting of the device 1 preventing a movement of the extremity 94. To this end, a patient 90 can be operatively connected to the device 1 according to the invention, especially to the first intervention means 3, preferably via the movement module 5. Subsequently, the force F(t) between the movement module 5 and an extremity 94 of the patient 90 can be measured with the aid of at least one force sensor 41; 51 during the execution of a movement cycle, that is to say a “step”, in a non-disrupted state. In the next step, the extremity 94, especially the leg 92 as shown here, is held securely by the first intervention means 3, preferably the movement module 5, that is to say is virtually fully prevented from carrying out its movement. Now, the force F(t) generated by the patient 90 to fight against the impediment by the first intervention means 3 and to nevertheless move can be measured with the aid of the force sensor or sensors 41; 51.

FIG. 5b shows an exemplary plot of the force F(t) exerted by the patient 90 on the device 1 from FIG. 5a, as measured by the latter, in the case of a non-disrupted movement of the extremity 94 and in the case of a disrupted, especially fully prevented, movement of the extremity 94; and FIG. 5 shows an exemplary plot of a flexion angle φ₍ₜ₎; corresponding to the performance of an individual movement analysis and movement therapy as shown in FIGS. 5a and 5b in exemplary fashion, as a function of time t. In this case, Fᵤₚ(t) and φᵤₚ(t) each describe the non-disrupted state and Fₚ(t) and φₚ(t) describe the disrupted state, in which the first intervention means 3 virtually fully prevents the movement. In the example shown here, the leg 92 of the patient 90 is kept stretched out in the disrupted state—the flexion angle φₚ(t) of the hip joint 95 consequently remains constant at 0° or 180° (depending on the definition of the zero) throughout the period t of the disruption P (=holding the leg 92 securely). The force Fₚ(t) with which the patient 90 works against the disruption P depends in turn on the current, individual state of health and can be compared with the measured values of the non-disrupted step Fᵤₚ(t) and/or with database of values of healthy humans or other patients.

The present invention relates to a device 1 for performing individual movement analysis and therapy on a patient, which device is characterized in that a control and analysis unit 6 is designed to control the first intervention means 3 in such a way that a first trajectory T₁ of the movement of an extremity 94 of the patient 90 is disrupted by a force exerted by a movement module 5 onto the extremity 94 of the patient 90; a response to this disruption P is measured via changed measured values from at least one force sensor 41; 51 and/or at least one angle sensor 52, and a new, second trajectory T₂ is calculated therefrom; and new control parameters for (further) controlling the first intervention means 3 are calculated from a comparison of the second trajectory T₂ with the first trajectory T₁ and/or a target trajectory Tₛₒₗₗ or the comparison of the disrupted measured values with the non-disrupted measured values. Advantageously, the device 1 according to the invention in particular facilitates the testing of patients in the early stage of their healing process in respect of the chances of success of possible therapy interventions, the development of an individualized therapy strategy and the tracking of the healing process even during the movement therapy. Within the scope of the individualized movement analysis and movement therapy facilitated by the device 1 according to the invention, it is possible in particular to deduce the type of therapy intervention or therapy component to which the respective patient 90 is most likely to react as a “responder”, that is to say with positive progress of the rehabilitation, by way of a targeted addition or omission of different therapy components.

### LIST OF REFERENCE SIGNS

1 Device
11 Device for reversibly connecting the device (1), especially the first intervention means (3), to a bed (80), in particular to a hospital bed that can be driven into the vertical
3 First intervention means
30 Rehabilitation mechanism
40 Foot module
41 Force sensor
5 Movement module
50 Knee module
51 Force sensor
52 Angle sensor
6 Control and analysis unit
61 Means for performing electromyography
7 Second intervention means
71 Means for generating a vibration
72 Means for performing electromyostimulation
73 Means for generating visual stimuli
74 Means for generating acoustic stimuli
75 Means for scheduled administration of a medicament
80 Bed, especially hospital bed that can be driven into the vertical
90 Patient
91 Foot
92 Leg
93 Knee joint
94 Extremity
95 Hip joint
Tₛₒₗₗ; T₁; T₂; . . . Trajectory
F(t) Force between the movement module (5) and an extremity (94) of the patient (90)
Fᵤₚ(t); Fₚ(t) Force (F(t)), non-disrupted and disrupted, respectively
Hᵤₚ(n); Hₚ(n) Step height; non-disrupted and disrupted, respectively
φᵤₚ(t); φₚ(t) Flexion angle of the hip joint (95), non-disrupted and disrupted, respectively
T Time
n Step number
P Disruption

1-9. (canceled)

10. A device for performing an individual movement analysis and movement therapy on a patient, the device comprising:
a first intervention device constructed as a rehabilitation mechanism configured for an automated rehabilitation movement of extremities, at least of joints, muscles and tendons of legs, of a patient according to plan;
at least one movement module configured to be brought into operative connection with the extremities of the patient;
said at least one movement module including at least one of:
at least one force sensor for measuring an absolute value of a force between said at least one movement module and an extremity of the patient, or
at least one angle sensor for measuring a direction of the force between said at least one movement module and the extremity of the patient; and
a control and analysis unit for controlling said first intervention device and analysis of a trajectory of the movement of the extremity of the patient calculated from actual values of at least one of said at least one force sensor or said at least one angle sensor;
said control and analysis unit is configured:
to control said first intervention device to disrupt a first trajectory of the movement of the extremity of the patient by a force exerted by said at least one movement module on the extremity of the patient;
to measure a response to the disruption by way of altered measured values of at least one of said at least one force sensor or said at least one angle sensor and calculate a new, second trajectory therefrom; and
to calculate new control parameters for further control of said first intervention device from a comparison of the second trajectory with at least one of the first trajectory or a target trajectory or a comparison of the disrupted and non-disrupted measured values of at least one of said at least one force sensor or said at least one angle sensor.

11. The device according to claim 10, wherein:
said rehabilitation mechanism has at least one of one knee module or a foot module forming said at least one movement module;
said one knee module configured to be brought into operative connection with knee joints of the patient and said one knee module including at least one of:
said at least one force sensor for measuring an absolute value of a force between said one knee module and a knee joint of the patient, or
said at least one angle sensor for measuring a direction of the force between the knee module and the knee joint of the patient; and
said foot module configured for accommodating feet of the patient and said foot module including said at least one force sensor for measuring an absolute value of a force between said foot module and each foot of the patient.

12. The device according to claim 10, which further comprises at least one second intervention device for interaction with the patient, said at least one second intervention device being configured to interchange control data with said control and analysis unit.

13. The device according to claim 12, wherein said at least one second intervention device is configured to interact with the body of the patient or with at least one of a leg, a knee joint or a foot of the patient.

14. The device according to claim 12, wherein said at least one second intervention device is a device for at least one of generating a vibration or performing electromyostimulation.

15. The device according to claim 12, wherein said at least one second intervention device is a device for generating at least one of visual stimuli or acoustic stimuli.

16. The device according to claim 12, wherein said at least one second intervention device is a device for scheduled administration of a medicament.

17. The device according to claim 10, wherein said control and analysis unit includes a device for performing electromyography.

18. The device according to claim 10, which further comprises a device for reversibly connecting the device or said first intervention device to a bed or to a hospital bed configured to be driven vertically.
